(19) United States
(12) Patent Application Publication
(10) Pub. No.: US 2025/0146993 A1
(43) Pub. Date: May 8, 2025

(54) METHOD AND SYSTEM FOR IDENTIFYING AMBIENT BACKGROUND LEVEL USING FEATURE MASK PROCESSING

(71) Applicant: SeekOps Inc., Austin, TX (US)

(72) Inventor: Kyle William Dawson, Austin, TX (US)

(21) Appl. No.: 18/938,985

(22) Filed: Nov. 6, 2024

Related U.S. Application Data
(60) Provisional application No. 63/547,416, filed on Nov. 6, 2023.

(51) Int. Cl. G01N 33/00; G01M 3/04

(52) U.S. Cl. CPC G01N 33/0027; G01M 3/04

(57) ABSTRACT

Systems, devices, and methods including: measuring, by a gas sensor, gas concentrations; transmitting, by the gas sensor, concentration signals corresponding to the measured gas concentrations to a processor; evaluating, by a feature identification module of the processor, each individual concentration signal with a plurality of criteria, where the evaluating includes identifying if the individual concentration signal is a peak signal based on whether a plurality of values extracted from the individual concentration signal according to the plurality of criteria are within thresholds and tagging the individual concentration signal as a feature if the individual concentration signal is the peak signal; and calculating, by an ambient background level calculator module of the processor, an ambient background gas level by removing the measured gas concentration corresponding to the concentration signal set as a feature from an original background level.

### CROSS-REFERENCE TO RELATED APPLICATION

This application claims the priority benefit of U.S. Provisional Patent Application Ser. No. 63/547,416 filed Nov. 6, 2023, incorporated herein by reference in its entirety.

### FIELD OF ENDEAVOR

The invention relates to gas measurement, and more particularly to identifying trace gas concentrations over an ambient background level.

### BACKGROUND

Methane (CH4) is an odorless and colorless naturally occurring organic molecule, which is present in the atmosphere at average ambient levels of approximately 1.85 ppm as of 2018 and is projected to continually climb. Methane is a powerful greenhouse gas, a source of energy (i.e., methane is flammable), and an explosion hazard, and so detection of methane is of utility to scientists as well as engineers. While methane is found globally in the atmosphere, a significant amount is collected or “produced” through anthropogenic processes including exploration, extraction, and distribution of petroleum resources as a component in natural gas. Natural gas, an odorless and colorless gas, is a primary fuel used to produce electricity and heat. The main component of natural gas is typically methane, and the concentration of methane in a stream of natural gas can range from about 70% to 90%. The balance of the gas mixture in natural gas consists of longer chain hydrocarbons, including ethane, propane, and butane, typically found in diminishing mole fractions that depend on the geology of the earth from which the gas is extracted. Once extracted from the ground, natural gas is processed into a product that must comply with specifications for both transport, taxation, and end-use in burners; specification of processed ‘downstream’ natural gas product control for the composition of the gas, so as to protect transport lines from corrosion and ensure proper operation of burners and turbines. While extraction of natural gas is one of the main sources of methane in the atmosphere, major contributors of methane also include livestock farming (i.e., enteric fermentation) and solid waste and wastewater treatment (i.e., anaerobic digestion). Anaerobic digestion and enteric fermentation gas products consist primarily of methane and lack additional hydrocarbon species.

### SUMMARY

A method embodiment may include: determining, by a feature mask processing, if a concentration value from a concentration signal is a feature; determining, by the feature mask processing, if a normalized power from the concentration signal is a feature; determining, by the feature mask processing, if a normalized wavelet probability density function (PDF) from the concentration signal is a feature; determining, by the feature mask processing, if a centroid of a normalized wavelet cumulative distribution function (CDF) from the concentration signal is a feature; determining, by the feature mask processing, if a rate-of-change threshold from the concentration signal is a feature; determining, by the feature mask processing, if a vertical velocity threshold from the concentration signal is a feature; and determining, by the feature mask processing, if a persistence threshold from the concentration signal is a feature.

Additional method embodiments may include: generating a bitmask based on each determined feature, where a 1 indicates a feature and a 0 indicates no feature.

Additional method embodiments may include: decoding a whole number into an on or off state of the thresholds comprising the bitmask.

A method embodiment may include: measuring, by at least one gas sensor, gas concentrations at a plurality of locations; transmitting, by the gas sensor, a plurality of concentration signals corresponding to the measured gas concentrations to a processor; evaluating, by a feature identification module of the processor, each individual concentration signal of the concentration signals with a plurality of criteria, wherein the step for evaluating includes identifying if the individual concentration signal is a peak signal based on whether a plurality of values extracted from the individual concentration signal according to the plurality of criteria are within thresholds and tagging the individual concentration signal as a feature if the individual concentration signal is the peak signal; and calculating, by an ambient background level calculator module of the processor, an ambient background gas level by removing the measured gas concentration corresponding to the concentration signal set as a feature from an original background level.

In additional method embodiments, the step for evaluating includes: extracting, by a criteria value extraction module of the processor, a plurality of values from the individual concentration signal according to the plurality of criteria.

In additional method embodiments, the step for evaluating each individual concentration signal includes: determining, by a value on and off setting module of the processor, if the plurality of values are within the thresholds for the plurality of criteria, respectively, and setting the values as on if the value is within a threshold; generating, by an integer value generation module of the processor, an integer value of the individual concentration signal by adding up value contribution of all the values that are set as on; and tagging, by a feature tagging module, the concentration signals as a feature or a no-feature, wherein a concentration signal is tagged as a feature if the integer value of the concentration signal is above a threshold.

In additional method embodiments, the step for evaluating each individual concentration signal is performed based on a feature bitmask including a plurality of bits by a feature mask processing module, and wherein the values extracted from the individual concentration signal respectively corresponds to the plurality of bits in the feature bitmask. In additional method embodiments, the value contribution by each bit in the feature bitmask is calculated in a binary number, which is represented as a power of 2. In additional method embodiments, the values from the extracted from the individual concentration signal includes at least two of: a concentration value; a normalized power; a normalized wavelet probability density function (PDF); a centroid of a normalized wavelet cumulative distribution function (CDF); a concentration rate-of-change; a vertical velocity; and a persistence.

In additional method embodiments, in the step for setting the values as on, the concentration value is set as on if the concentration value is above a predetermined threshold, the normalized power is set as on if the normalized power is above a predetermined threshold, the normalized wavelet PDF is set as on if the normalized wavelet PDF is above a predetermined threshold, the centroid of a normalized wavelet CDF is set as on if the centroid of a normalized wavelet CDF is above a predetermined threshold, the concentration rate-of-change is set as on if the concentration rate-of-change is above a predetermined threshold, the vertical velocity is set as on if the vertical velocity is below a predetermined threshold; and the persistence is set as on if an individual concentration signal directly before a current individual concentration signal is tagged as a feature.

In additional method embodiments, the feature bitmask is based on out of 7 bits, and wherein the seventh bit position is corresponding to a concentration value, the sixth bit position is corresponding to a normalized power; the fifth bit position is corresponding to a normalized wavelet probability density function (PDF); the fourth bit position is corresponding to a centroid of a normalized wavelet cumulative distribution function (CDF); the third bit position is corresponding to a concentration rate-of-change; the second bit position is corresponding to a vertical velocity; and the first bit position is corresponding to a persistence.

In additional method embodiments, the at least one gas sensor is mounted on an unmanned aerial system (UAS), and the step of measuring the gas concentrations at the plurality of positions is performed while the UAS is during the flight. In additional method embodiments, the step for measuring the gas concentrations is performed at the plurality of locations over a cross section of a gas plume within a flux plane geometry.

In additional method embodiments, the step for measuring gas concentrations is performed repeatedly at the same locations in a regular basis. Additional method embodiments may include: displaying, by a display, a normally-distributed surface of the measured gas concentrations.

Another method embodiment may include: measuring, by at least one gas sensor, a gas concentration; determining, by a feature mask processing module of a processor, if a concentration value from a concentration signal corresponding to the gas concentration is a feature; determining, by the feature mask processing module, if a normalized power from the concentration signal is a feature; determining, by the feature mask processing module, if a normalized wavelet probability density function (PDF) from the concentration signal is a feature; determining, by the feature mask processing module, if a centroid of a normalized wavelet cumulative distribution function (CDF) from the concentration signal is a feature; determining, by the feature mask processing module, if a concentration rate-of-change from the concentration signal is a feature; determining, by the feature mask processing module, if a vertical velocity from the concentration signal is a feature; and determining, by the feature mask processing module, if a persistence from the concentration signal is a feature.

Additional method embodiments may include: generating a bitmask based on each determined feature, wherein a 1 indicates a feature and a 0 indicates no feature.

Additional method embodiments may include: decoding a whole number into an on or off state of thresholds comprising the bitmask.

A system embodiment may include: at least one gas sensor configured to measure gas concentrations at a plurality of locations and transmit a plurality of concentration signals corresponding to the measured gas concentrations to a processor; and the processor configured to communicate with the at least one gas sensor and including: a feature identification module configured to evaluate each individual concentration signal of the concentration signals with a plurality of criteria by identifying if the individual concentration signal is a peak signal based on whether a plurality of values extracted from the individual concentration signal according to the plurality of criteria are within thresholds and tagging the individual concentration signal as a feature if the individual concentration signal is the peak signal; and an ambient background level calculator module configured to calculate an ambient background gas level by removing the measured gas concentration corresponding to the concentration signal set as a feature from an original background level.

In additional system embodiments, the feature identification module includes: a criteria value extraction module configured to extract a plurality of values from the individual concentration signal according to the plurality of criteria; a value on and off setting module configured to determine if the plurality of values are within the thresholds for the plurality of criteria, respectively, and set the values as on if the value is within a threshold; an integer value generation module configured to generate an integer value of the individual concentration signal by adding up value contribution of all the values that are set as on; and a feature tagging module configured to tag the concentration signals as a feature or a no-feature, wherein a concentration signal is tagged as a feature if the integer value of the concentration signal is above a threshold.

In additional system embodiments, the feature identification module includes a feature mask processing module configured to generate a feature bitmask including a plurality of bit, and wherein the values extracted from the individual concentration signal respectively corresponds to the plurality of bits in the feature bitmask.

In additional system embodiments, the values from the extracted from the individual concentration signal includes: a concentration value, a normalized power, a normalized wavelet probability density function (PDF), a centroid of a normalized wavelet, cumulative distribution function (CDF), a concentration rate-of-change, a vertical velocity, and a persistence; and wherein the concentration value is set as on if the concentration value is above a predetermined threshold, the normalized power is set as on if the normalized power is above a predetermined threshold, the normalized wavelet PDF is set as on if the normalized wavelet PDF is above a predetermined threshold, the centroid of a normalized wavelet CDF is set as on if the centroid of a normalized wavelet CDF is above a predetermined threshold, the concentration rate-of-change is set as on if the concentration rate-of-change is above a predetermined threshold, the vertical velocity is set as on if the vertical velocity is below a predetermined threshold; and the persistence is set as on if an individual concentration signal directly before a current individual concentration signal is tagged as a feature.

In additional system embodiments, the at least one gas sensor is mounted on an unmanned aerial system (UAS), and the gas concentrations at the plurality of positions is measured while the UAS is during the flight.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1A depicts a diagram of a system for identifying an ambient background level using feature mask processing, according to one embodiment;

FIG. 1B depicts a flow chart of a method for identifying an ambient background level using feature mask processing, according to one embodiment;

FIG. 1C depicts a flow chart of steps for evaluating each individual concentration signal with a plurality of criteria shown in FIG. 1B, according to one embodiment;

FIG. 1D depicts a flow chart of steps for evaluating each individual concentration signal with a plurality of criteria shown in FIG. 1B, according to one embodiment;

FIG. 1E depicts a normally-distributed surface with z axis representing concentration in arbitrary units and x and y axes representing distance and altitude-along-plane in arbitrary units, according to one embodiment;

FIG. 2A depicts a probability density function of power with reference to period and normalized to a maximum of 1 and normalized concentration signal, according to one embodiment;

FIG. 2B depicts a continuous wavelet transform of normalized power spectra of gaus2 wavelet convolved with the concentration signal, according to one embodiment;

FIG. 3 depicts a shape of the gaus2 mother wavelet, according to one embodiment;

FIG. 4 depicts a value of power variance with reference to time and as a function of wavelet period, according to one embodiment;

FIG. 5 depicts a cumulative distribution function at each time step and normalized to a maximum value of 1, according to one embodiment;

FIG. 6A depicts a result of a feature mask processing output for altitude colored by an altitude mask, according to one embodiment;

FIG. 6B depicts a result of a feature mask processing output for concentration time series colored by altitude mask, according to one embodiment;

FIG. 6C depicts a result of a feature mask processing output for concentration time series colored by feature mask, according to one embodiment;

FIG. 6D depicts a result of a feature mask processing output for the difference in the original minus the updated background signal, according to one embodiment;

FIG. 6E depicts a result of a feature mask processing output colored by bitmask value, according to one embodiment;

FIG. 6F depicts a result of a feature mask processing output decoded bitmask, according to one embodiment;

FIG. 7 illustrates an example top-level functional block diagram of a computing device embodiment, according to one embodiment;

FIG. 8 shows a high-level block diagram and process of a computing system for implementing an embodiment of the system and process, according to one embodiment;

FIG. 9 shows a block diagram and process of an exemplary system in which an embodiment may be implemented, according to one embodiment;

FIG. 10 depicts a cloud-computing environment for implementing an embodiment of the system and process disclosed herein, according to one embodiment; and

FIG. 11 depicts a system for detecting trace gasses, according to one embodiment.

### DETAILED DESCRIPTION

The following description is made for the purpose of illustrating the general principles of the embodiments discloses herein and is not meant to limit the concepts disclosed herein. Further, particular features described herein can be used in combination with other described features in each of the various possible combinations and permutations. Unless otherwise specifically defined herein, all terms are to be given their broadest possible interpretation including meanings implied from the description as well as meanings understood by those skilled in the art and/or as defined in dictionaries, treatises, etc.

The present system and method are configured to identify an ambient background gas level by using a processing that identifies characteristics in a target gas (e.g., methane) concentration time series that indicate a presence of a feature over the ambient background level.

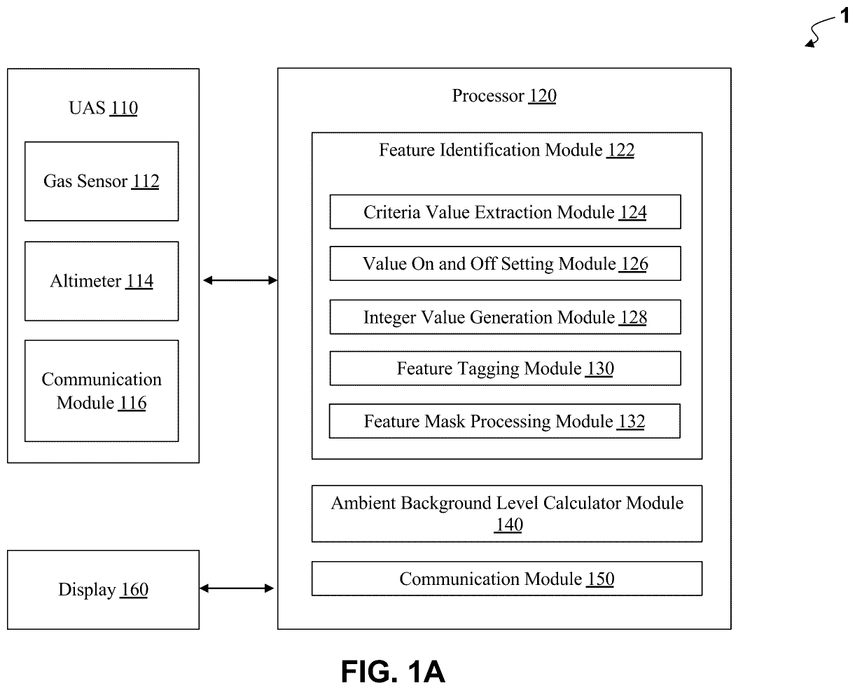

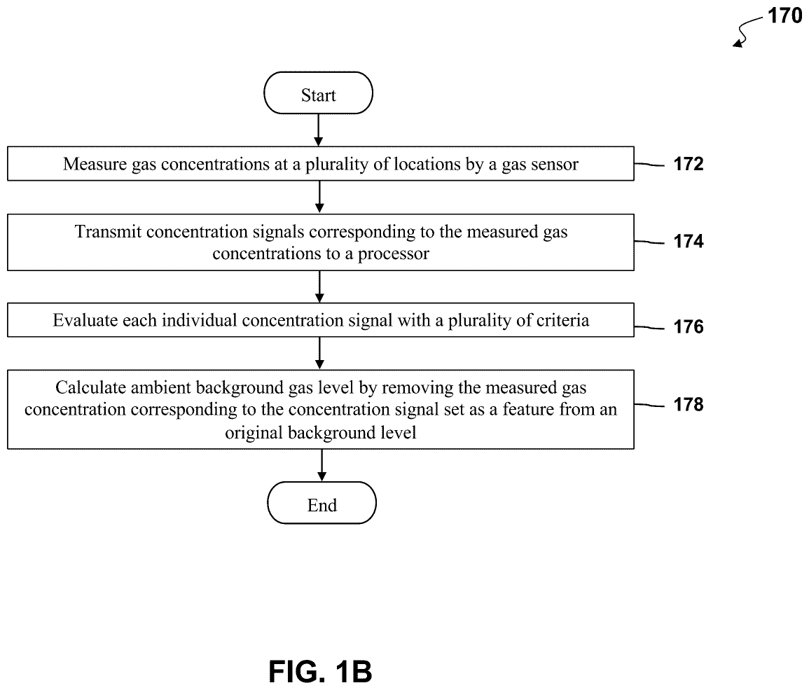

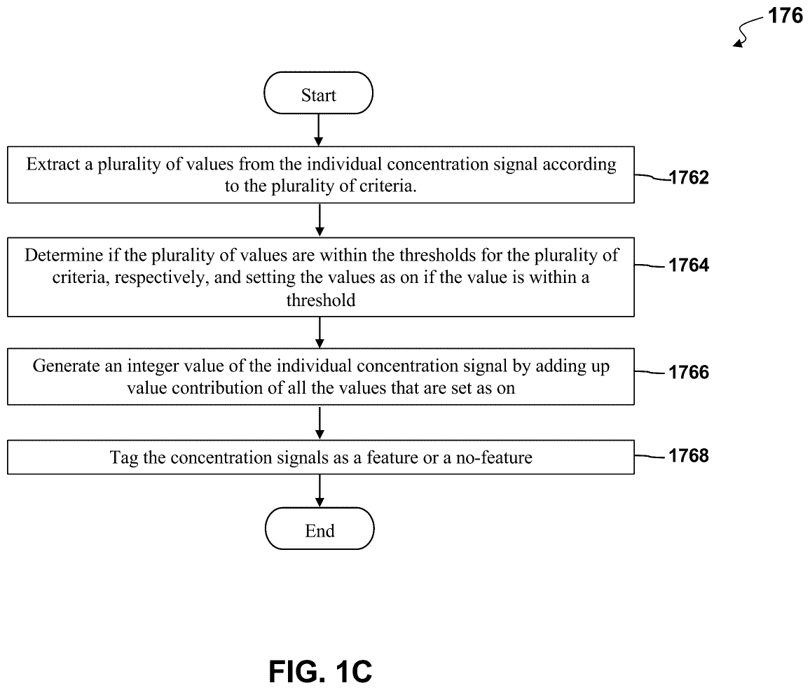

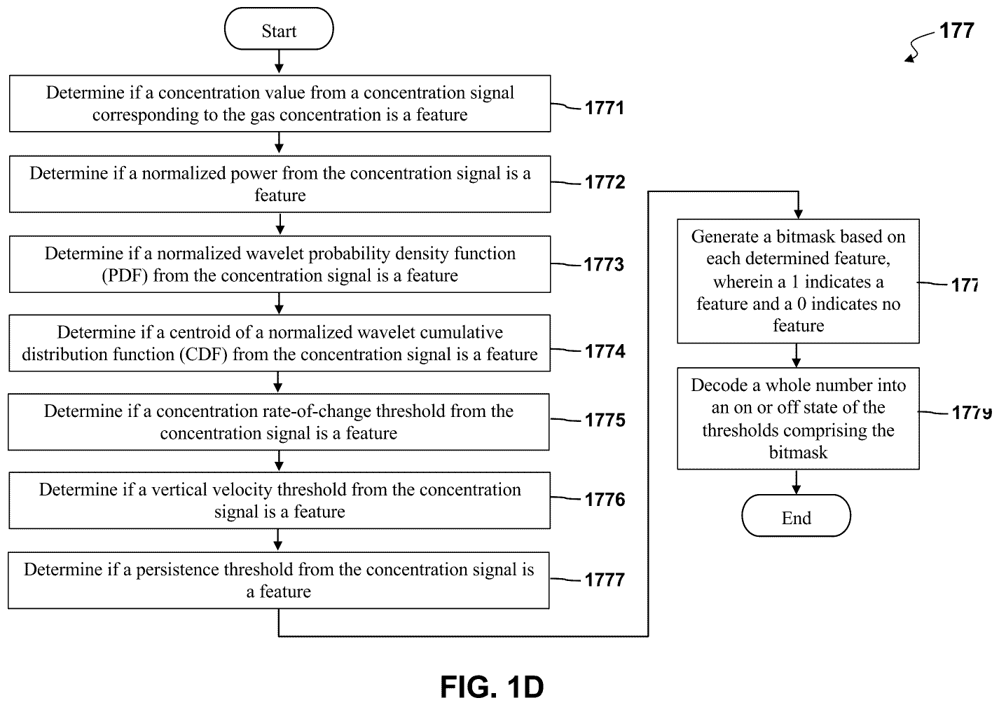

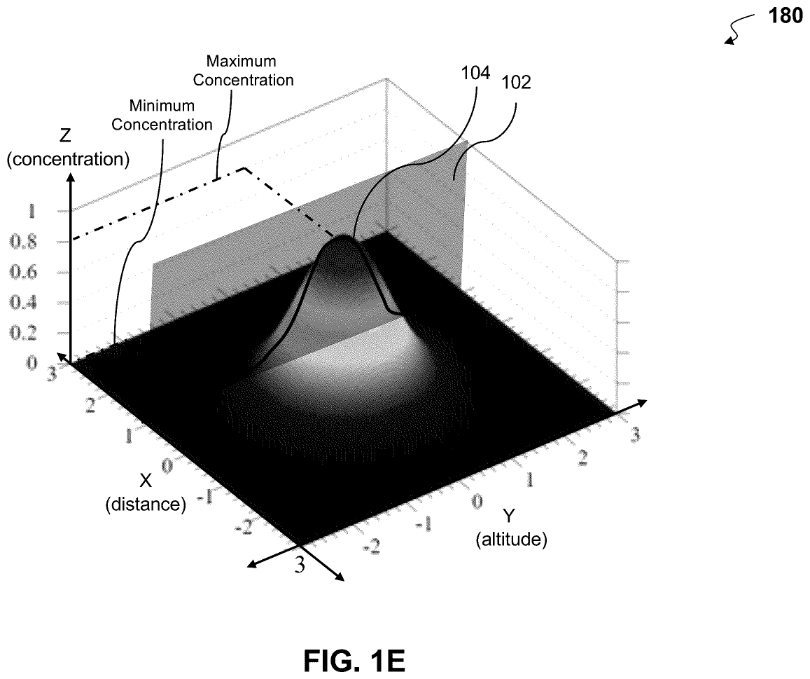

FIG. 1A depicts a diagram of a system for identifying an ambient background level using feature mask processing, according to one embodiment. Referring to FIG. 1A, the system 100 may comprise an unmanned aerial system (UAS) 110 including a gas sensor 112, a processor 120, and a display 160. Specifically, the UAV 112 may include at least one gas sensor 112 configured to measure gas concentrations at a plurality of locations. The at least one gas sensor 112 may be mounted on the UAS 110, and the step of measuring the gas concentrations at the plurality of positions by the gas sensor 112 may be performed while the UAS 110 is during the flight. In some embodiments, the gas concentrations may be measured at the plurality of locations over a cross section of a gas plume within a flux plane geometry. The UAS 110 may further include altimeter 114 configured to measure the altitude of the UAS 110 and/or the gas sensor 112. The UAS 110 and/or the gas sensor 112 may be configured to communicate with the processor 120 using a communication module 116. The gas sensor 112 may be configured to transmit a plurality of concentration signals corresponding to the measured gas concentrations to the processor 120 through the communication module 116. In some embodiments, the gas concentrations may be measured repeatedly at the same locations in a regular basis to analyze concentration rate of change over time.

The processor 120 may include a feature identification module 122, an ambient background level calculator module 140, and a communication module 150. Specifically, the feature identification module 122 may be configured to receive the measured gas concentrations data from the gas sensor 112 through the communication module 150. The feature identification module 122 may then be configured to evaluate each individual concentration signal of the concentration signals with a plurality of criteria by identifying if the individual concentration signal is a peak signal based on whether a plurality of values extracted from the individual concentration signal according to the plurality of criteria are within thresholds and tagging the individual concentration signal as a feature if the individual concentration signal is the peak signal.

This concentration signal evaluation processing by the feature identification module 122 may be run as a preprocessing step to an ambient background level calculator processing by the ambient background level calculator module 140. The concentration signal evaluation processing function may tag the concentration signals as feature or not-feature so as to remove peaks, i.e., “features”, from the concentration signals since the peaks would bias the estimate of the ambient background level calculator.

Then, the ambient background level calculator module 140 may be configured to calculate an ambient background gas level by removing the measured gas concentration corresponding to the concentration signal set as a feature from an original background level. In this way, the disclosed system and method can arrive at a better ambient background signal that is free from the influences of prolonged methane enhancements, such as when measuring methane in certain environments such as landfills.

In some embodiments, the feature identification module 122 may include: a criteria value extraction module 124, a value on and off setting module 126, an integer value generation module 128, a feature tagging module 130, and a feature mask processing module 132. The criteria value extraction module 124 may be configured to extract a plurality of values from the individual concentration signal according to the plurality of criteria. In some embodiments, a plurality of values from the individual concentration signal according to the plurality of criteria may include at least two of: a concentration value of the individual concentration signal, a normalized power of the individual concentration signal; a normalized wavelet probability density function (PDF) of the individual concentration signal; a centroid of a normalized wavelet cumulative distribution function (CDF) of the individual concentration signal; a concentration rate-of-change of the individual concentration signal; and a vertical velocity of the individual concentration signal; and a persistence of the individual concentration signal.

The plurality of values from each individual concentration signal may be evaluated based on a feature bitmask including a plurality of bits by a feature mask processing module 132 of the feature identification module 122. The output of the feature mask processing for the measured gas concentration signals may be a tuple of three variables, each of the same shape as the input concentration signal. The first variable is a mask of Is and Os where 1=feature and 0=no-feature. The second variable is a bitmask that has a whole number that can be decoded into the different flags that were activated for that particular datapoint of the concentration signal. The third variable is a sequentially-numbered vector where each number indicates an altitude level. The second variable of the output is the “bitmask” portion of the processing. The bitmask will be described in detail below with a lookup table for decoding whole numbers of features into their respective activated flags.

The values extracted from the individual concentration signal respectively may correspond to the plurality of bits in the feature bitmask. The value contribution by each bit in the feature bitmask is calculated in a binary number, which is represented as a power of 2. In some embodiments, the feature bitmask may be based out of 7 flags (or bits) but is not limited thereto. Other flag or bit numbers are possible and contemplated. In the case where there are 7 flags available, the 7 bits can represent 2⁷=128 combinations with an integer value from 0 to 127.

The value on and off setting module 126 may be configured to determine if the plurality of values are within the thresholds for the plurality of criteria, respectively, and set the values as on if the value is within a threshold. In this case, the concentration value is set as on if the concentration value is above a predetermined threshold; the normalized power is set as on if the normalized power is above a predetermined threshold; the normalized wavelet PDF is set as on if the normalized wavelet PDF is above a predetermined threshold; the centroid of a normalized wavelet CDF is set as on if the centroid of a normalized wavelet CDF is above a predetermined threshold; the concentration rate-of-change is set as on if the concentration rate-of-change is above a predetermined threshold; the vertical velocity is set as on if the vertical velocity is below a predetermined threshold; and the persistence is set as on if an individual concentration signal directly before a current individual concentration signal is tagged as a feature.

Each combination of the 7 bits may correspond to a different combination of flags being turned on or off, and a maximum integer value of 2⁷−1=127 is for all flags being turned on. Flags are labeled as “on=1” or “off=0” from right to left, starting with 0-indexed for the rightmost bit (the first bit position) and 6-indexed for the leftmost bit (the seventh bit position).

The integer value generation module 128 may be configured to generate an integer value of the individual concentration signal by adding up value contribution of all the values that are set as on. Each individual concentration signal may be evaluated based on a feature bitmask including a plurality of bits by the feature mask processing module 132, and the values extracted from the individual concentration signal respectively may correspond to the plurality of bits in the feature bitmask. In this case, the value contribution by each bit in the feature bitmask may be calculated in a binary number, which is represented as a power of 2 (2\hat{ }index). In the above example, the first bit position (0-index) represents 2⁰, the second bit position (1-index) represents 2¹, the third bit position (2-index) represents 2², the fourth bit position (3-index) represents 2³, the fifth bit position (4-index) represents 2⁴, the sixth bit position (5-index) represents 2⁵, and the seventh bit position (6-index) represents 2⁶. The integer value of the individual concentration signal may represent the combined value of all flags set in the bitmask, and thus the integer value of the bitmask may be determined by adding up the value contributions of all the flags that are set to 1.

For example, a bit string with flags in the first (0-index), fourth (3-index), and sixth (5-index) position looks like “0101001” and has an integer value of 2⁰+2³+2⁵=41. The bit furthest to the left contributes a larger value to the integer number than the bit furthest to the right. Therefore, the flag criteria are ordered similarly, in way that reflects this same weighting.

The table of flags for the feature bitmask processing may be given below:

| | | Criteria | | Bit |
|---|---|---|---|---|
| | Bit | Threshold | Criteria | Value |
| Feature Flag | Position | Default | Unit | x: 2^{x} |
| Concentration Value [vmr] | 7 | >5e⁻⁶ | mol mol⁻¹ | 6 |
| Normalized power [on a scale of | 6 | >0.5 | fraction | 5 |
| 0-1] at the dilation value where | | | out of 1 | |
| the maximum of power variance | | | | |
| occurs | | | | |
| Normalized Wavelet PDF | 5 | >0.25 | fraction | 4 |
| integrated with reference to | | | out of 1 | |
| dilation (period) | | | | |
| Centroid of the Normalized | 4 | >0.5 | fraction | 3 |
| Wavelet CDF integrated with | | | out of 1 | |
| reference to translation (time) | | | | |
| and taken at variance maximum | | | | |
| *Change in concentration with | 3 | >5e−7 | mol mol⁻¹ | 2 |
| time | | | s⁻¹ | |
| *Change in altitude with time | 2 | <1.0 | ms⁻¹ | 1 |
| (i.e., vertical velocity) | | | | |
| Persistence of feature. If the | 1 | True | unitless | 0 |
| datapoint directly before the | | | | |
| current datapoint is tagged as a | | | | |
| feature, then this information | | | | |
| is persisted | | | | |

In the above table, the feature flag marked with asterisk “*” may indicate a signal that has been simplified using a discrete wavelet decomposition function with the wavelet= ‘rbio2.2’ mother wavelet.

In some embodiments, the thresholds may be revised by the user and the bitmask processing may be run again with the new thresholds which would produce a different result. The thresholds provided herein and in the above table are examples. Other thresholds are possible and contemplated.

The feature tagging module 130 may be configured to tag the concentration signals as a feature or a no-feature. The concentration signal may be tagged as a feature if the integer value of the concentration signal is above a predetermined threshold. In this way, the concentration signals may be tagged as feature or no-feature and then the concentration signal tagged as features may be removed from the original background level to calculate an ambient background gas level that is free from the influences of prolonged trace gas enhancements, such as when measuring trace gas (e.g., methane) in certain environments such as landfills.

The display 160 may be configured to display a normally-distributed surface of the measured gas concentrations as shown in FIG. 1E.

FIG. 1B depicts a flow chart of a method for identifying an ambient background level using feature mask processing, according to one embodiment. Referring to FIG. 1B, the method 170 may comprise: measuring, by at least one gas sensor, gas concentrations at a plurality of locations (step 172); transmitting, by the gas sensor, a plurality of concentration signals corresponding to the measured gas concentrations to a processor (step 174); evaluating, by a feature identification module of the processor, each individual concentration signal of the concentration signals with a plurality of criteria, wherein the step for evaluating includes identifying if the individual concentration signal is a peak signal based on whether a plurality of values extracted from the individual concentration signal according to the plurality of criteria are within thresholds and tagging the individual concentration signal as a feature if the individual concentration signal is the peak signal (step 176); and calculating, by an ambient background level calculator module of the processor, an ambient background gas level by removing the measured gas concentration corresponding to the concentration signal set as a feature from an original background level (step 178).

FIG. 1C depicts a flow chart of steps for evaluating each individual concentration signal with a plurality of criteria in the step 176 shown in FIG. 1B, according to one embodiment. Referring to FIG. 1C, the step of valuating each individual concentration signal with a plurality of criteria may include the steps of: extracting, by a criteria value extraction module of the processor, a plurality of values from the individual concentration signal according to the plurality of criteria (step 1762); determining, by a value on and off setting module of the processor, if the plurality of values are within the thresholds for the plurality of criteria, respectively, and setting the values as on if the value is within a threshold (step 1764); generating, by an integer value generation module of the processor, an integer value of the individual concentration signal by adding up value contribution of all the values that are set as on (step 1766); and tagging, by a feature tagging module, the concentration signals as a feature or a no-feature, wherein a concentration signal is tagged as a feature if the integer value of the concentration signal is above a threshold (step 1768).

FIG. 1D depicts a flow chart of steps for evaluating each individual concentration signal with a plurality of criteria shown in FIG. 1B, according to one embodiment. Referring to FIG. 1D, the step 177 of evaluating each individual concentration signal with a plurality of criteria may include steps of: measuring, by at least one gas sensor, a gas concentration (step 172); determining, by a feature mask processing module of a processor, if a concentration value from a concentration signal corresponding to the gas concentration is a feature (step 1771); determining, by the feature mask processing module, if a normalized power from the concentration signal is a feature (step 1772); determining, by the feature mask processing module, if a normalized wavelet probability density function (PDF) from the concentration signal is a feature (step 1773); determining, by the feature mask processing module, if a centroid of a normalized wavelet cumulative distribution function (CDF) from the concentration signal is a feature (step 1774); determining, by the feature mask processing module, if a concentration rate-of-change from the concentration signal is a feature (step 1775); determining, by the feature mask processing module, if a vertical velocity from the concentration signal is a feature (step 1776); and determining, by the feature mask processing module, if a persistence from the concentration signal is a feature (step 1777). The steps for evaluating each individual concentration signal with a plurality of criteria may further include steps of: generating a bitmask based on each determined feature, where a 1 indicates a feature and a 0 indicates no feature (step 1778) and decoding a whole number into an on or off state of thresholds comprising the bitmask (step 1779).

FIG. 1E depicts a normally-distributed surface 180 with z axis representing gas concentration in arbitrary units and x and y axes representing distance and altitude-along-plane in arbitrary units.

### Example BitMask Decode

In some embodiments, the bitmask value is a result of adding up the value contribution of all the flags that are tagged as “on,” which means that a bit string for the bitmask value is already known as a predetermined factor.

In one example, decoding a bitmask value of 42 results in “0101010” transposed into a [7×1] matrix. Specifically, the decimal value 42 in binary string is “00101010” (8 bits), and to represent the binary string as a [7×1] output matrix, the last 7 bits of the binary representation “0101010” may be extracted. The decoded value represents that flags corresponding to bit positions 6, 4, and 2 are True and flags corresponding to bit positions 7, 5, 3, and 1 are False. The size and shape of the output matrix may be arranged as [7×N] for easy plotting as seen in the graphical representation of FIG. 1.

### Bitmask Criteria Descriptions

### Concentration Value

The concentration threshold may be the first mask criteria. A datapoint may be tagged with the seventh bit as “on” when the concentration value signal exceeds this threshold in units of mole fraction.

This threshold may be changed when toggling between different gas species detection, such as methane and carbon dioxide trace gas detection. In this case, a first gas species (e.g., carbon dioxide) has background concentrations that are two orders of magnitude larger than those of a second gas species (e.g., methane). Other thresholds may be used for other trace gases.

### Normalized Power, Normalized Wavelet PDF, and CDF Wavelet Transform (Continuous and Discrete)

The normalized power may be the second mask criteria, the normalized wavelet probability density function (PDF) may be the third mask criteria, and the normalized cumulative distribution function (CDF) may be the fourth mask criteria. The power, the normalized PDF, and the normalized CDF may be based on continuous wavelet transforms (CWT) and discrete wavelet transforms (DWT).

In some embodiments, gas concentrations may be surveyed over a cross section of a gas plume within a Flux Plane geometry. In this case, if the plume cross-section is viewed in a 3D space, it would resemble a normally-distributed surface like FIG. 1. Slices of FIG. 1 along an x-axis and a y-axis may correspond to the cross section of the gas plume, and a center of gas mass exists between more-or-less symmetric tails on either side of curve, i.e., a Gaussian shaped curve.

Slices 102 of FIG. 1 along a z-axis and a y-axis represent a Gaussian curve 104 of gas concentration as the gas sensor disposed on an unmanned aerial system (UAS) climbs in altitude along the y-axis. An ideal signal of gas concentration may be depicted by a Gaussian curve where the minimum z-axis is the concentration background, and the maximum z-axis is the concentration center of mass. However, there is noise and potentially multiple sources of trace gas within a single altitude level that can obscure this feature.

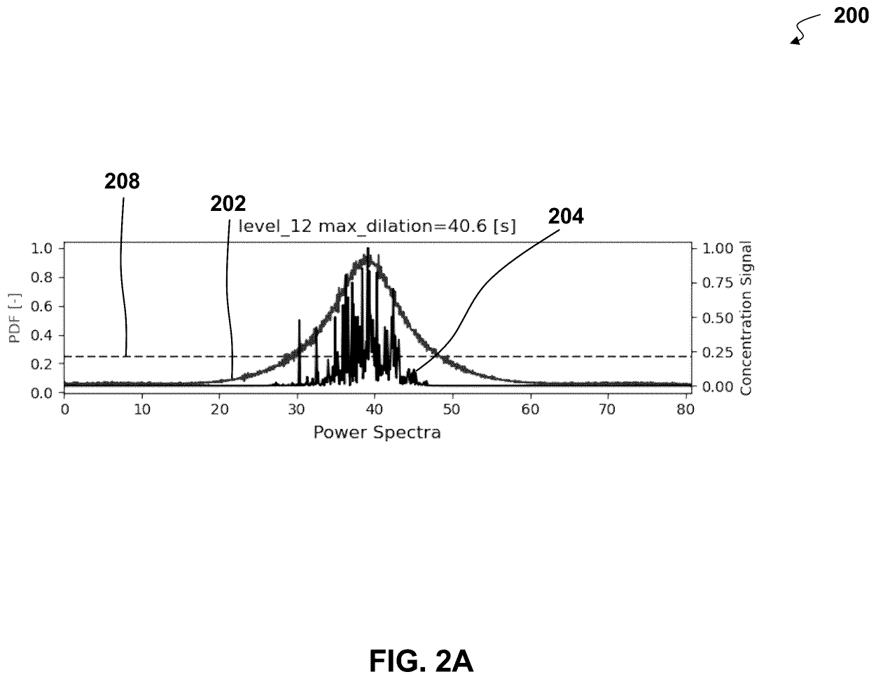

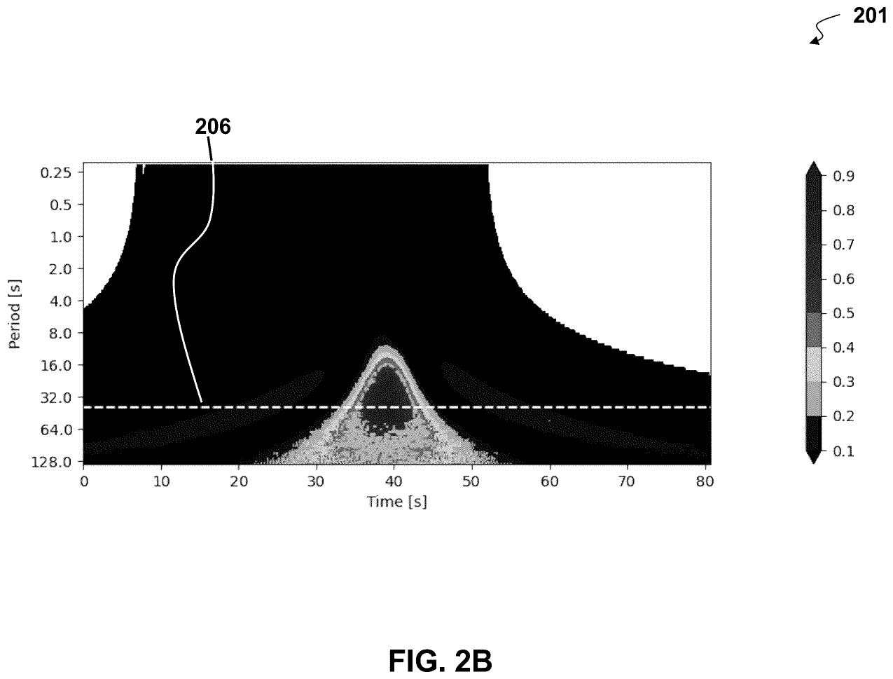

FIG. 2A depicts a graph 200 for a probability density function (PDF) of power with reference to period, which is normalized to a maximum of 1, and normalized concentration signal. The probability density function (PDF) of power with reference to period, which is normalized to a maximum of 1, is shown as a curve 202. The normalized concentration signal is shown as a curve 204. The threshold value is shown as a dashed line 208.

A continuous wavelet transform takes a wavelet of a known shape (which may also be called the mother wavelet) and convolves it along the signal aiming to pull out the pattern that is most similar to the mother wavelet. The period/frequency of the continuous wavelet may be stretched or shrunk (i.e., is dilated), and the center of the wavelet may be moved along the time-series (i.e., is translated) to generate a power spectra as a function of period and time. Maximum power occurs where the mother wavelet and the signal are coincident.

An example power spectra for a modeled concentration signal is shown in FIGS. 2A-2B. This method is similar to Fourier Transform (FFT) with one key difference. In FFT, the entire signal is decomposed into frequency representation so that the timing of particular amplitude spikes needs to be constant and repeatable, otherwise the reconstruction of the signal will be off. In Continuous Wavelet Transform (CWT), the wavelet convolving with the signal at different scale gives frequency/spectral information, but also local temporal information due to the translation along the time-series.

FIG. 2B depicts a graph 201 for a continuous wavelet transform (CWT) of normalized power spectra of gaus2 wavelet convolved with the concentration signal. The normalized signal concentration is shown as a curve 204. The power spectra at the dilation of maximum variance are shown by a dashed line 206.

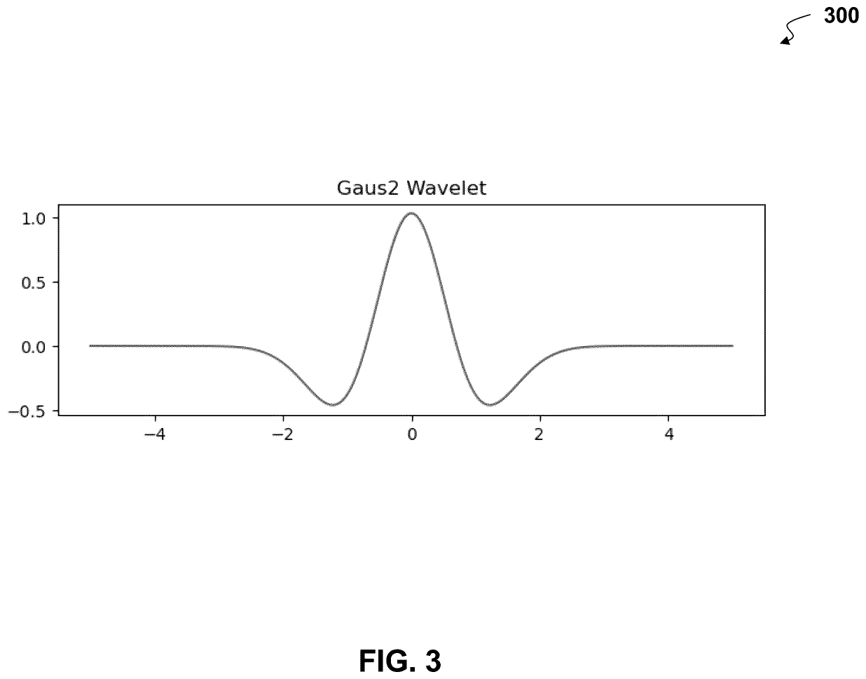

FIG. 3 depicts a graph 300 for a shape of the gaus 2 mother wavelet. The wavelet that was convolved with the concentration signal is called a gaus2 wavelet and has the shape shown in FIG. 3.

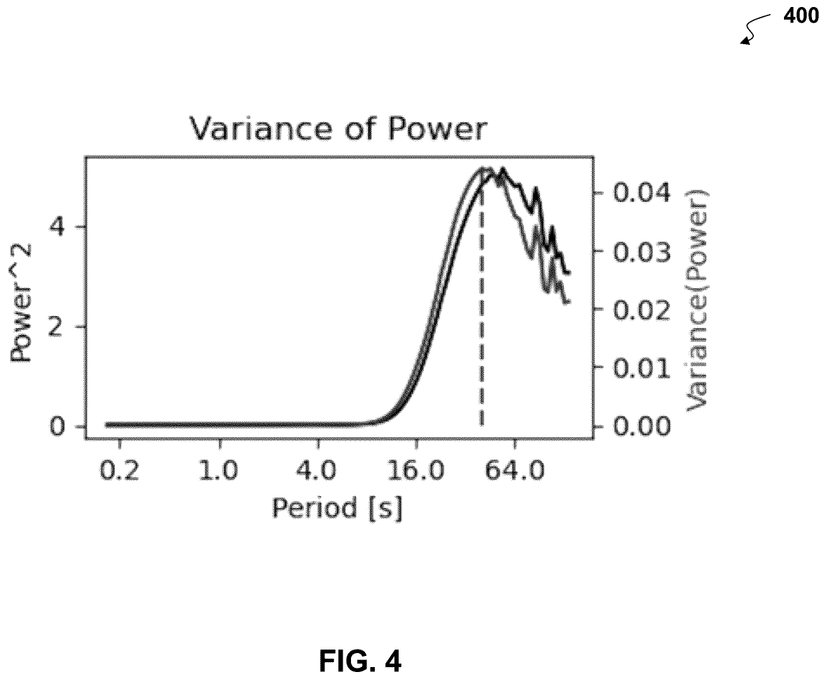

FIG. 4 depicts a graph 400 for a value of power variance with reference to time and as a function of wavelet period (i.e., dilation).

Extracting Information about Features

The power spectra that is shown in FIG. 2B must be converted into information about a feature location. Processing to extract feature information using CWT may use the Haar wavelet. The Haar wavelet helps to extract the boundary of the largest change in a signal such as in a transition of a scalar in the atmospheric mixed layer versus the free troposphere. The disclosed system and methos may use the gaus2 wavelet and other mother wavelets that exhibit a similar shape to the feature that is desired to emphasize.

Once the wavelet is selected and the power spectra produced, the disclosed system and method can identify the period where the maximum variance of the power spectra occurs. This region should be coincident with an emphasized peak, followed by near-zero values where there is no peak. In some embodiments, exceptions may occur when the scale is poorly selected. In some embodiments, the scale may be constrained to be a minimum of the Nyquist rate of 0.2 sec (f=5 Hz) and a maximum of 4 times the time period of the signal segment which appears to be appropriate for most applications.

The power spectra at the dilation of maximum variance (shown to the left in FIG. 4) is shown by a dashed line 206 in FIG. 2B. Furthermore, the contour shows the boundary inside which the normalized power is >0.5 (or whatever threshold the user selects). The PDF of the power spectra along the dashed line 206 is shown in FIG. 2A of the top panel by the curve 202.

The combination of the region of the PDF above a threshold value (the dashed line 208 of FIG. 2) and the yellow contour of the power identify the regions of the concentration signal that contribute to the second and third flags of the feature mask.

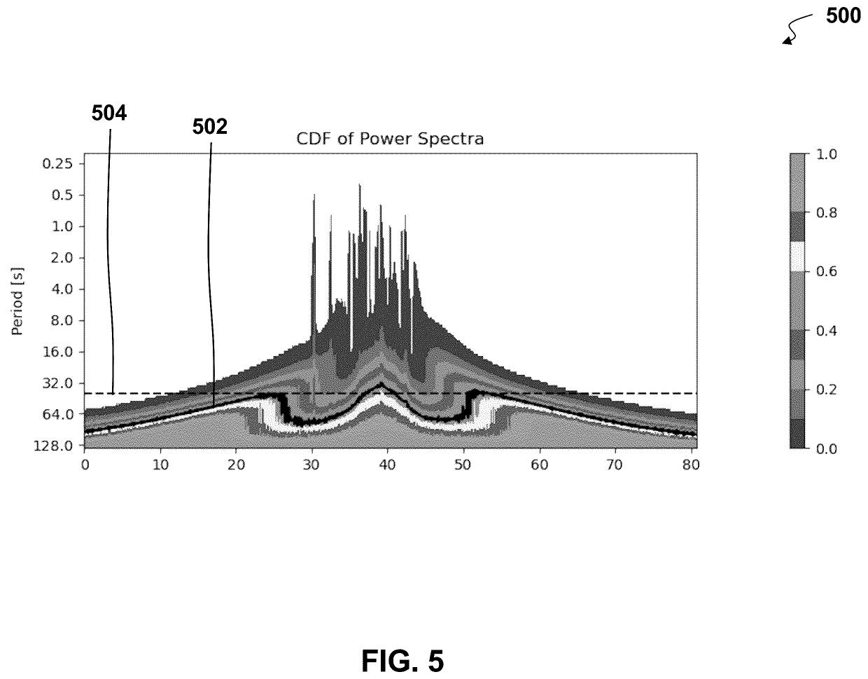

FIG. 5 depicts a graph 500 for a cumulative distribution function (CDF) at each time step and normalized to a maximum value of 1. The solid contour 502 identifies the user-selected threshold value (in this case 0.5) where the cross section of the dilation at maximum variance (dashed line 504).

The fourth flag of the feature mask examines the normalized CDF of the power spectra at each time step at the dilation value of maximum variance. FIG. 5 shows the model dataset of this feature. In this case, the default threshold value of 0.5 may be very restrictive and may be lowered to a more appropriate value of 0.3, for example. Other dilation values are possible and contemplated.

### Rate-of-Change Thresholds

The fifth and sixth mask criteria may be the concentration rate-of-change threshold and the vertical velocity threshold. The background concentration may not change drastically with time while intersecting a plume is a large concentration change with time. The absolute value of this change (increasing or decreasing) may flag this feature when it is greater than a threshold (in this case 0.5 ppm per second). This signal may be smoothed using a discrete wavelet transform to remove signal noise for a clearer representation of this feature.

The vertical velocity should be near 0 for each altitude level. Changing vertical velocity greater than a threshold (in this case 1 meter per second) may be considered unstable and therefore not contribute to the feature mask. No plots may be provided for these thresholds.

### Persistence Threshold

The seventh mask criteria may be a persistence threshold, The persistence threshold considers the state of the mask at the previous timestamp (t−δt). If the state of the mask just prior was tagged as a feature, then a “true” flag persists, otherwise, a “false” flag persists. This is intended to provide hysteresis to the processing.

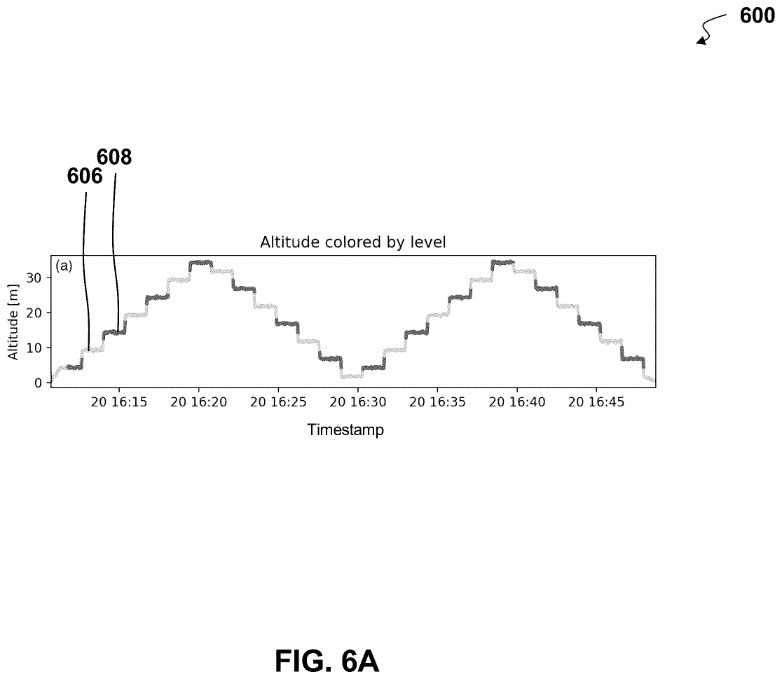

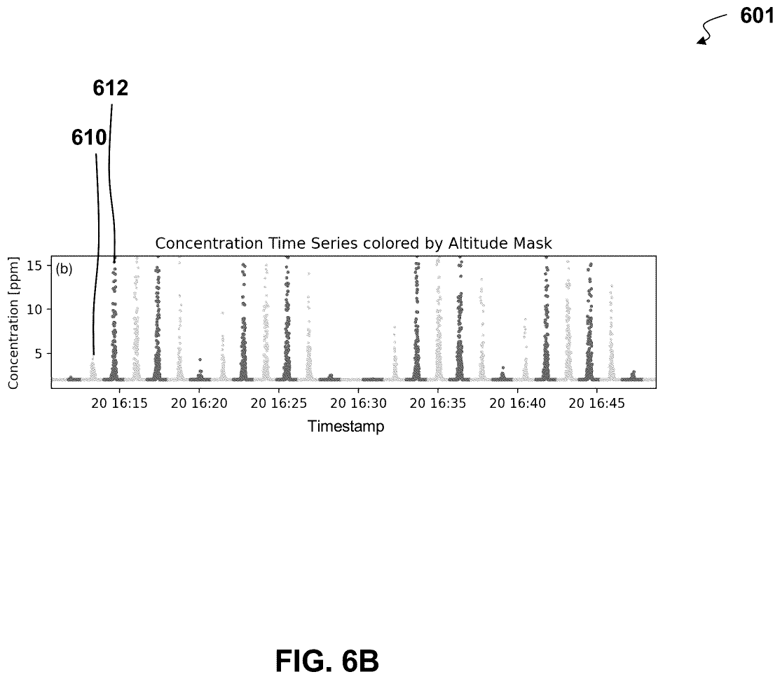

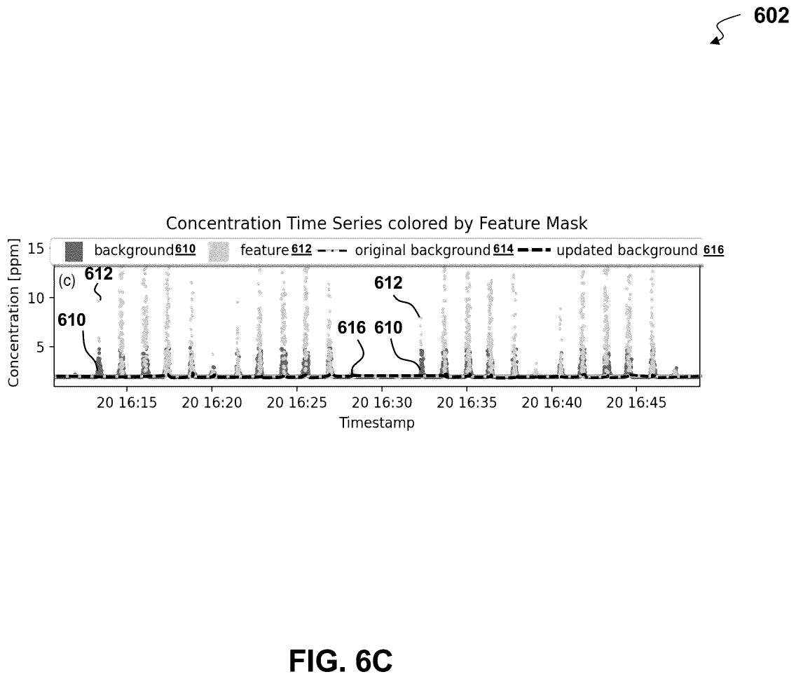

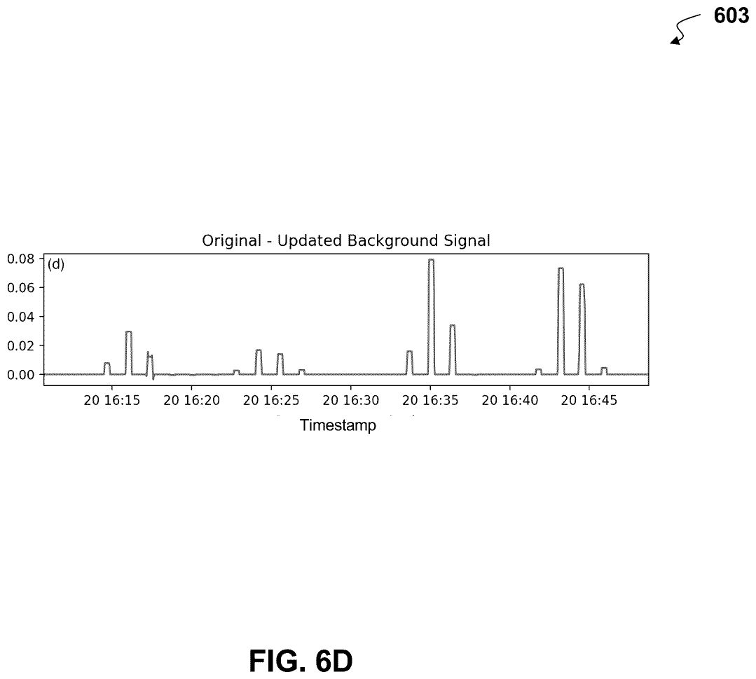

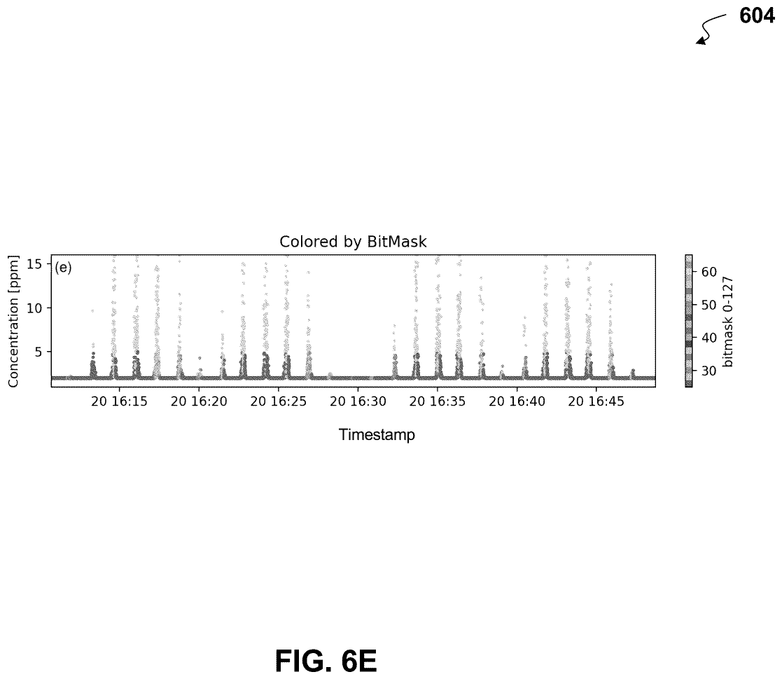

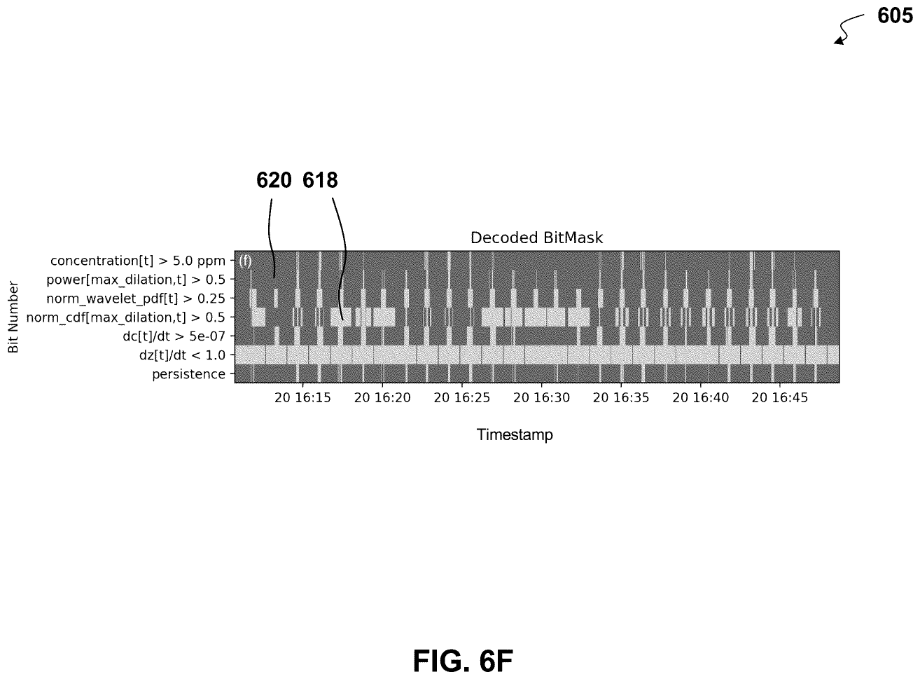

FIGS. 6A-6E depict results of a feature mask processing.

FIG. 6A depicts a graph 600 for a result of a feature mask processing output for altitude colored by an altitude mask over time. FIG. 6A shows the result of the altitude segmentation. Alternating colors 608, 608 indicate different levels.

FIG. 6B depicts a graph 601 for a result of a feature mask processing output for concentration time series colored by altitude mask over time. FIG. 6B shows the concentration time series where alternating colors 610, 612 correspond to the different altitude levels shown in FIG. 6A.

FIG. 6C depicts a graph 602 for a result of a feature mask processing output for concentration time series colored by feature mask. In the graph in FIG. 6C, dark color spots 610 are labeled as feature=0 (i.e., background) and light color spots 612 are labeled as feature=1 (i.e., features). The original background estimate is shown in dashed dot line 614 and the updated background estimate is shown in dashed line 616.

FIG. 6D depicts a graph 603 for a result of a feature mask processing output for the difference in the original background estimate (614, FIG. 6C) minus the updated background signal (616, FIG. 6C). Because differences in the original background estimates are hard to discern in some cases, FIG. 6D shows the difference of the original background estimate and the updated background estimates.

FIG. 6E depicts a graph 604 for a result of a feature mask processing output colored by bitmask value. FIG. 6E shows the value of the bitmask in integer format.

FIG. 6F depicts a diagram 605 for a result of a feature mask processing output decoded bitmask. A python code snippet, such as the one shown below, may be used to decode this value into its respective on/off flags. In the plot shown in FIG. 6F, the “On” flags are indicated by light area 618 and “off” flags by dark area 620.

```
    import numpy as np
“””
input: ndarray [1 × N] of bitmask integers (i.e., output[1] of feature_mask
processing)
output: [7 × N] matrix of int 0s and 1s. Top row is bit 7, Bottom row is bit
1.
“””
def decode_bitmask(feature_bitmask: np.ndarray):
bitmask_array = np.zeros([len(feature_bitmask),7],dtype=int)
for idx,bits in enumerate(feature_bitmask):
bitmask_array[idx,:] = [*format(bits,′#09b′)[2:][::−1]]
return bitmask_array.T
>> sample_input = np.array([42],dtype=int)
>> decode_bitmask(sample_input)
>> array([[0.],
  [1.],
  [0.],
  [1.],
  [0.],
  [1.],
  [0.]])
```

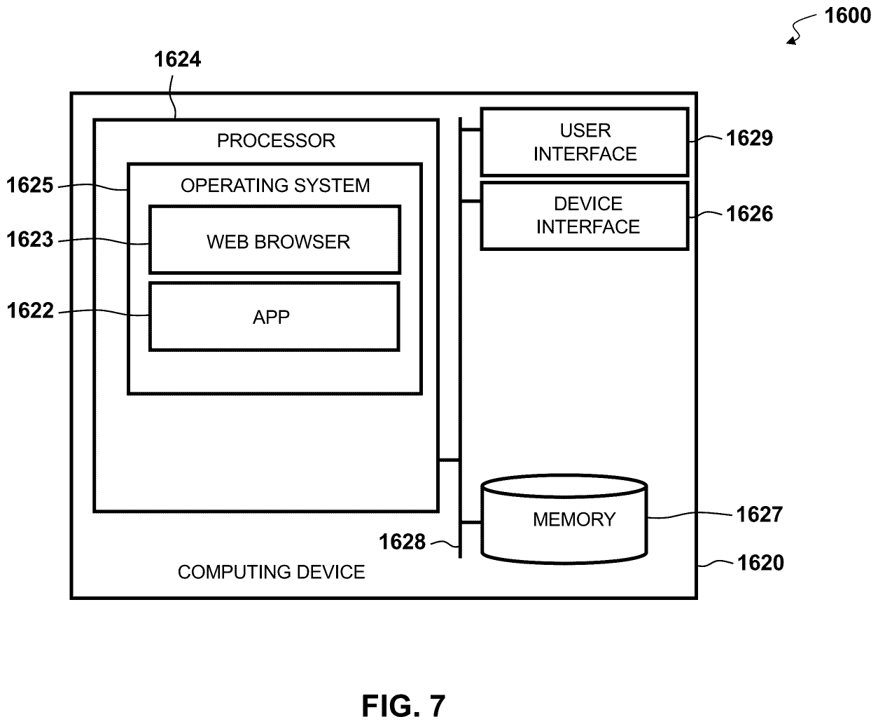

FIG. 7 illustrates an example of a top-level functional block diagram of a computing device embodiment 1600. The example operating environment is shown as a computing device 1620 comprising a processor 1624, such as a central processing unit (CPU), addressable memory 1627, an external device interface 1626, e.g., an optional universal serial bus port and related processing, and/or an Ethernet port and related processing, and an optional user interface 1629, e.g., an array of status lights and one or more toggle switches, and/or a display, and/or a keyboard and/or a pointer-mouse system and/or a touch screen. Optionally, the addressable memory may, for example, be: flash memory, eprom, and/or a disk drive or other hard drive. These elements may be in communication with one another via a data bus 1628. In some embodiments, via an operating system 1625 such as one supporting a web browser 1623 and applications 1622, the processor 1624 may be configured to execute steps of a process establishing a communication channel and processing according to the embodiments described above.

System embodiments include computing devices such as a server computing device, a buyer computing device, and a seller computing device, each comprising a processor and addressable memory and in electronic communication with each other. The embodiments provide a server computing device that may be configured to: register one or more buyer computing devices and associate each buyer computing device with a buyer profile; register one or more seller computing devices and associate each seller computing device with a seller profile; determine search results of one or more registered buyer computing devices matching one or more buyer criteria via a seller search component. The service computing device may then transmit a message from the registered seller computing device to a registered buyer computing device from the determined search results and provide access to the registered buyer computing device of a property from the one or more properties of the registered seller via a remote access component based on the transmitted message and the associated buyer computing device; and track movement of the registered buyer computing device in the accessed property via a viewer tracking component. Accordingly, the system may facilitate the tracking of buyers by the system and sellers once they are on the property and aid in the seller's search for finding buyers for their property. The figures described below provide more details about the implementation of the devices and how they may interact with each other using the disclosed technology.

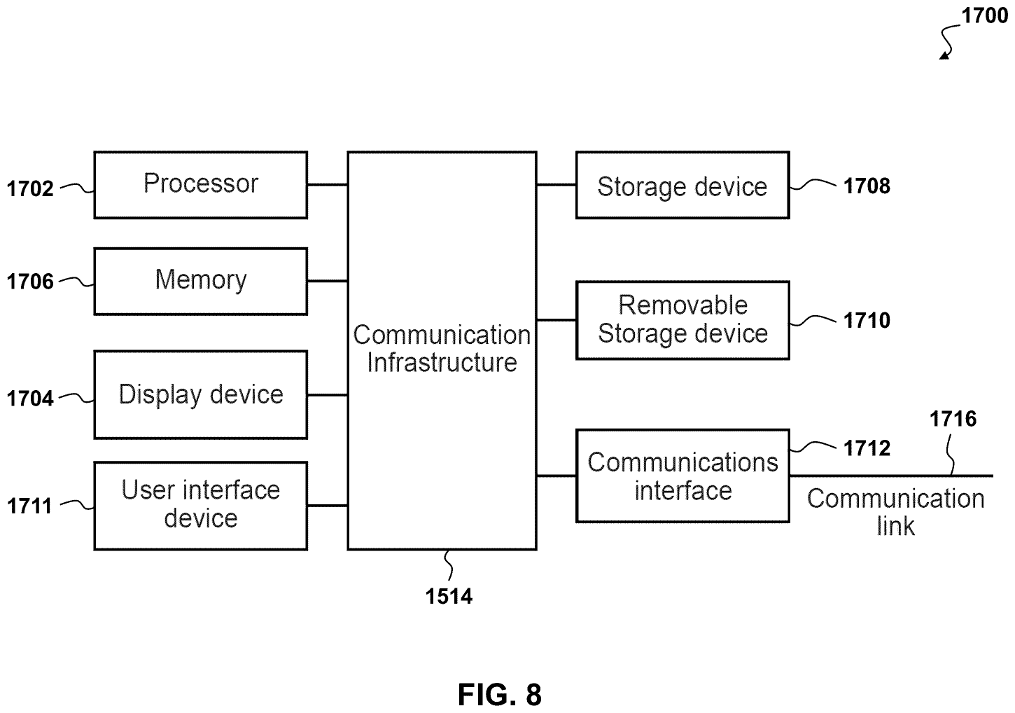

FIG. 8 is a high-level block diagram 1700 showing a computing system comprising a computer system useful for implementing an embodiment of the system and process, disclosed herein. Embodiments of the system may be implemented in different computing environments. The computer system includes one or more processors 1702, and can further include an electronic display device 1704 (e.g., for displaying graphics, text, and other data), a main memory 1706 (e.g., random access memory (RAM)), storage device 1708, a removable storage device 1710 (e.g., removable storage drive, a removable memory module, a magnetic tape drive, an optical disk drive, a computer readable medium having stored therein computer software and/or data), user interface device 1711 (e.g., keyboard, touch screen, keypad, pointing device), and a communication interface 1712 (e.g., modem, a network interface (such as an Ethernet card), a communications port, or a PCMCIA slot and card). The communication interface 1712 allows software and data to be transferred between the computer system and external devices. The system further includes a communications infrastructure 1514 (e.g., a communications bus, cross-over bar, or network) to which the aforementioned devices/modules are connected as shown.

Information transferred via communications interface 1712 may be in the form of signals such as electronic, electromagnetic, optical, or other signals capable of being received by communications interface 1724, via a communication link 1716 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 1712. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

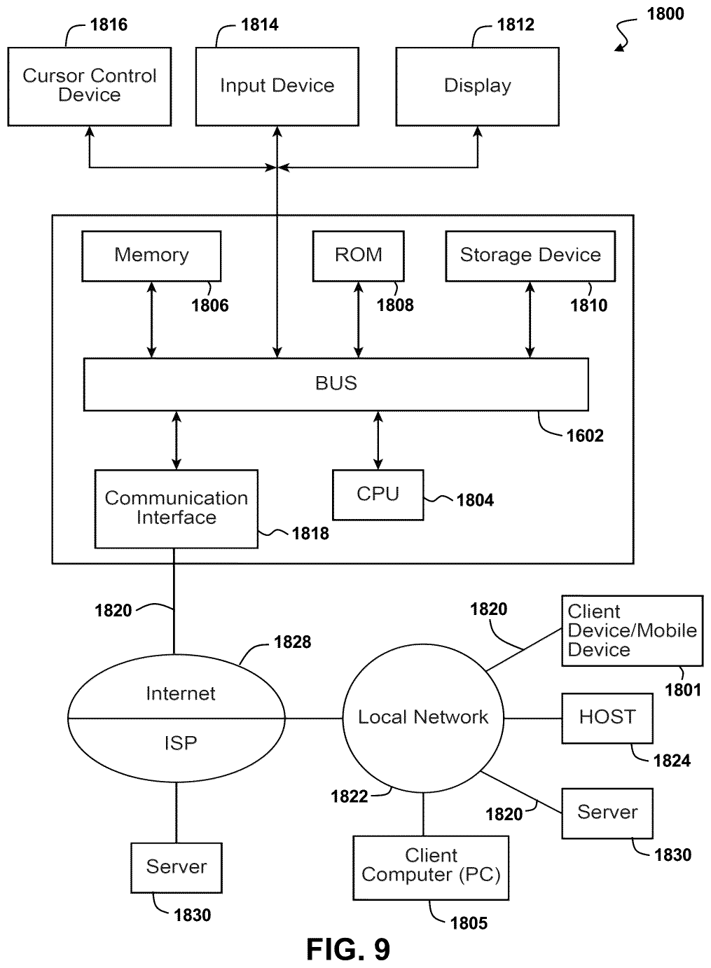

FIG. 9 shows a block diagram of an example system 1800 in which an embodiment may be implemented. The system 1800 includes one or more client devices 1801 such as consumer electronics devices, connected to one or more server computing systems 1830. A server 1830 includes a bus 1602 or other communication mechanism for communicating information, and a processor (CPU) 1804 coupled with the bus 1602 for processing information. The server 1830 also includes a main memory 1806, such as a random access memory (RAM) or other dynamic storage device, coupled to the bus 1602 for storing information and instructions to be executed by the processor 1804. The main memory 1806 also may be used for storing temporary variables or other intermediate information during execution or instructions to be executed by the processor 1804. The server computer system 1830 further includes a read only memory (ROM) 1808 or other static storage device coupled to the bus 1602 for storing static information and instructions for the processor 1804. A storage device 1810, such as a magnetic disk or optical disk, is provided and coupled to the bus 1602 for storing information and instructions. The bus 1602 may contain, for example, thirty-two address lines for addressing video memory or main memory 1806. The bus 1602 can also include, for example, a 32-bit data bus for transferring data between and among the components, such as the CPU 1804, the main memory 1806, video memory and the storage 1810. Alternatively, multiplex data/address lines may be used instead of separate data and address lines.

The server 1830 may be coupled via the bus 1602 to a display 1812 for displaying information to a computer user. An input device 1814, including alphanumeric and other keys, is coupled to the bus 1602 for communicating information and command selections to the processor 1804. Another type or user input device comprises cursor control 1816, such as a mouse, a trackball, or cursor direction keys for communicating direction information and command selections to the processor 1804 and for controlling cursor movement on the display 1812.

According to one embodiment, the functions are performed by the processor 1804 executing one or more sequences of one or more instructions contained in the main memory 1806. Such instructions may be read into the main memory 1806 from another computer-readable medium, such as the storage device 1810. Execution of the sequences of instructions contained in the main memory 1806 causes the processor 1804 to perform the process steps described herein. One or more processors in a multi-processing arrangement may also be employed to execute the sequences of instructions contained in the main memory 1806. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1804 for execution. Such a medium may take many forms, including but not limited to, non-volatile media, volatile media, and transmission media. Non-volatile media includes, for example, optical or magnetic disks, such as the storage device 1810. Volatile media includes dynamic memory, such as the main memory 1806. Transmission media includes coaxial cables, copper wire and fiber optics, including the wires that comprise the bus 1602. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1804 for execution. For example, the instructions may initially be carried on a magnetic disk of a remote computer. The remote computer can load the instructions into its dynamic memory and send the instructions over a telephone line using a modem. A modem local to the server 1830 can receive the data on the telephone line and use an infrared transmitter to convert the data to an infrared signal. An infrared detector coupled to the bus 1602 can receive the data carried in the infrared signal and place the data on the bus 1602. The bus 1602 carries the data to the main memory 1806, from which the processor 1804 retrieves and executes the instructions. The instructions received from the main memory 1806 may optionally be stored on the storage device 1810 either before or after execution by the processor 1804.

The server 1830 also includes a communication interface 1818 coupled to the bus 1602. The communication interface 1818 provides a two-way data communication coupling to a network link 1820 that is connected to the world wide packet data communication network now commonly referred to as the Internet 1828. The Internet 1828 uses electrical, electromagnetic, or optical signals that carry digital data streams. The signals through the various networks and the signals on the network link 1820 and through the communication interface 1818, which carry the digital data to and from the server 1830, are exemplary forms or carrier waves transporting the information.

In another embodiment of the server 1830, the communication interface 1818 is connected to a network 1822 via a communication link 1820. For example, the communication interface 1818 may be an integrated services digital network (ISDN) card or a modem to provide a data communication connection to a corresponding type of telephone line, which can comprise part of the network link 1820. As another example, the communication interface 1818 may be a local area network (LAN) card to provide a data communication connection to a compatible LAN. Wireless links may also be implemented. In any such implementation, the communication interface 1818 sends and receives electrical, electromagnetic, or optical signals that carry digital data streams representing various types of information.

The network link 1820 typically provides data communication through one or more networks to other data devices. For example, the network link 1820 may provide a connection through the local network 1822 to a host computer 1824 or to data equipment operated by an Internet Service Provider (ISP). The ISP in turn provides data communication services through the Internet 1828. The local network 1822 and the Internet 1828 both use electrical, electromagnetic, or optical signals that carry digital data streams. The signals through the various networks and the signals on the network link 1820 and through the communication interface 1818, which carry the digital data to and from the server 1830, are exemplary forms or carrier waves transporting the information.

The server 1830 can send/receive messages and data, including e-mail, program code, through the network, the network link 1820 and the communication interface 1818. Further, the communication interface 1818 can comprise a USB/Tuner and the network link 1820 may be an antenna or cable for connecting the server 1830 to a cable provider, satellite provider or other terrestrial transmission system for receiving messages, data and program code from another source.

The example versions of the embodiments described herein may be implemented as logical operations in a distributed processing system such as the system 1800 including the servers 1830. The logical operations of the embodiments may be implemented as a sequence of steps executing in the server 1830, and as interconnected machine modules within the system 1800. The implementation is a matter of choice and can depend on performance of the system 1800 implementing the embodiments. As such, the logical operations constituting said example versions of the embodiments are referred to for e.g., as operations, steps or modules.

Similar to a server 1830 described above, a client device 1801 can include a processor, memory, storage device, display, input device and communication interface (e.g., e-mail interface) for connecting the client device to the Internet 1828, the ISP, or LAN 1822, for communication with the servers 1830.

The system 1800 can further include computers (e.g., personal computers, computing nodes) 1805 operating in the same manner as client devices 1801, where a user can utilize one or more computers 1805 to manage data in the server 1830.

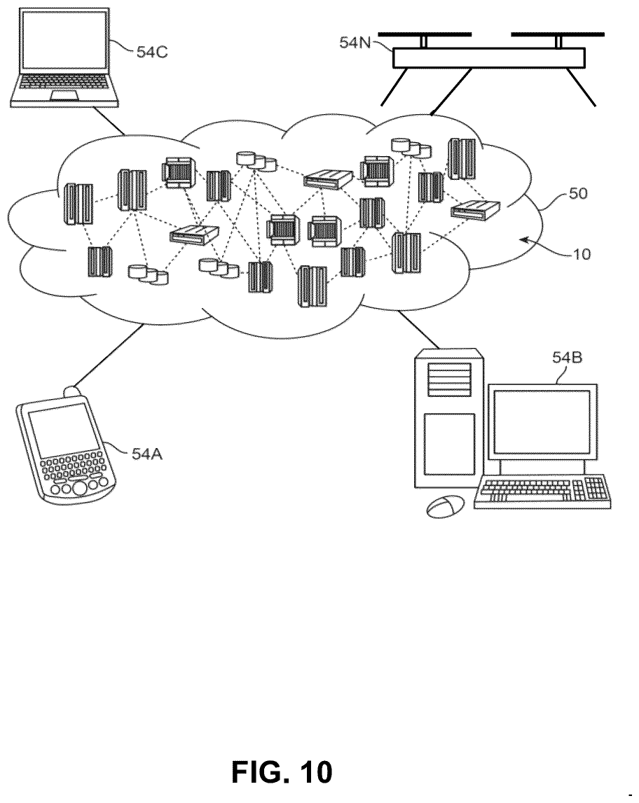

Referring now to FIG. 10, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or unmanned aerial system (UAS) 54N may communicate. The nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 10 are intended to be illustrative only and that computing nodes 10 and a cloud computing environment 50 can communicate with any type of computerized device over any type of network and/or network addressable connection (e.g., using a web browser).

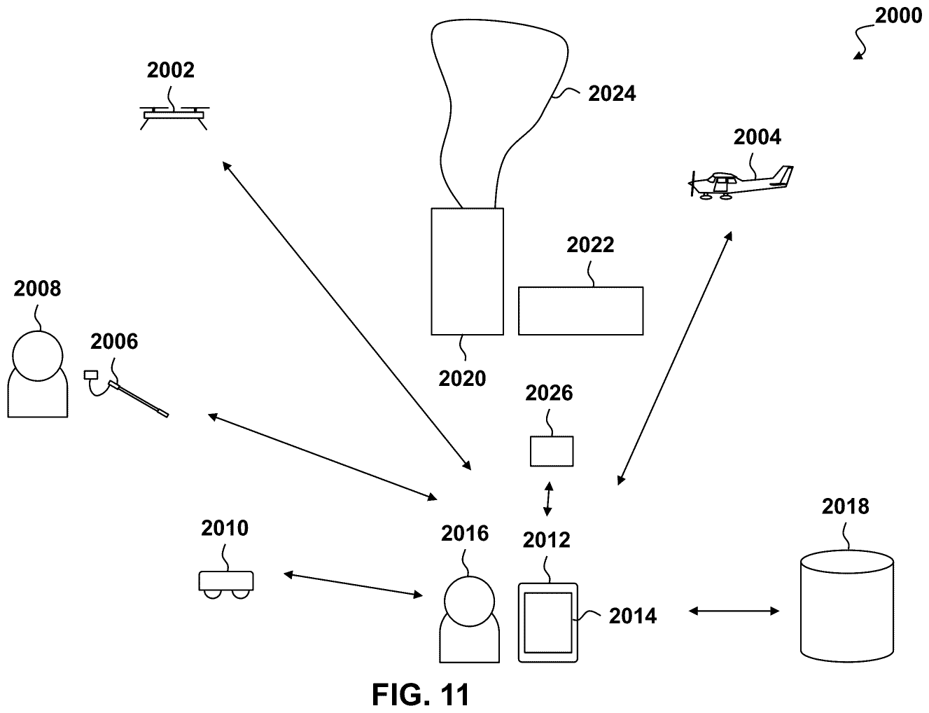

FIG. 11 depicts a system 2000 for detecting trace gasses, according to one embodiment. The system may include one or more trace gas sensors located in one or more vehicles 2002, 2004, 2006, 2010. The one or more trace gas sensors may detect elevated trace gas concentrations from one or more potential gas sources 2020, 2022, such as a holding tank, pipeline, or the like. The potential gas sources 2020, 2022 may be part of a large facility, a small facility, or any location. The potential gas sources 2020, 2022 may be clustered and/or disposed distal from one another. The one or more trace gas sensors may be used to detect and quantify leaks of toxic gases, e.g., hydrogen disulfide, or environmentally damaging gases, e.g., methane, sulfur dioxide) in a variety of industrial and environmental contexts. Detection and quantification of these leaks are of interest to a variety of industrial operations, such as oil and gas, chemical production, and painting. Detection and quantification of leaks is also of value to environmental regulators for assessing compliance and for mitigating environmental and safety risks. In some embodiments, the at least one trace gas sensor may be configured to detect methane. In other embodiments, the at least one trace gas sensor may be configured to detect sulfur oxide, such as SO, SO2, SO3, S7O2, S6O2, S2O2, and the like. A trace gas leak 2024 may be present in a potential gas source 2020. The one or more trace gas sensors may be used to identify the trace gas leak 2024 and/or the source 2020 of the trace gas leak 2024 so that corrective action may be taken.

The one or more vehicles 2002, 2004, 2006, 2010 may include an unmanned aerial vehicle (UAV) 2002, an aerial vehicle 2004, a handheld device 2006, and a ground vehicle 2010. In some embodiments, the UAV 2002 may be a quadcopter or other device capable of hovering, making sharp turns, and the like. In other embodiments, the UAV 2002 may be a winged aerial vehicle capable of extended flight time between missions. The UAV 2002 may be autonomous or semi-autonomous in some embodiments. In other embodiments, the UAV 2002 may be manually controlled by a user. The aerial vehicle 2004 may be a manned vehicle in some embodiments. The handheld device 2006 may be any device having one or more trace gas sensors operated by a user 2008. In one embodiment, the handheld device 2006 may have an extension for keeping the one or more trace gas sensors at a distance from the user 2008. The ground vehicle 2010 may have wheels, tracks, and/or treads in one embodiment. In other embodiments, the ground vehicle 2010 may be a legged robot. In some embodiments, the ground vehicle 2010 may be used as a base station for one or more UAVs 2002. In some embodiments, one or more aerial devices, such as the UAV 2002, a balloon, or the like, may be tethered to the ground vehicle 2010. In some embodiments, one or more trace gas sensors may be located in one or more stationary monitoring devices 2026. The one or more stationary monitoring devices may be located proximate one or more potential gas sources 2020, 2022. In some embodiments, the one or more stationary monitoring devices may be relocated.

The one or more vehicles 2002, 2004, 2006, 2010 and/or stationary monitoring devices 2026 may transmit data including trace gas data to a ground control station (GCS) 2012. The GCS may include a display 2014 for displaying the trace gas concentrations to a GCS user 2016. The GCS user 2016 may be able to take corrective action if a gas leak 2024 is detected, such as by ordering a repair of the source 2020 of the trace gas leak. The GCS user 2016 may be able to control movement of the one or more vehicles 2002, 2004, 2006, 2010 in order to confirm a presence of a trace gas leak in some embodiments.

In some embodiments, the GCS 2012 may transmit data to a cloud server 2018. In some embodiments, the cloud server 2018 may perform additional processing on the data. In some embodiments, the cloud server 2018 may provide third party data to the GCS 2012, such as wind speed, temperature, pressure, weather data, or the like.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention herein disclosed by way of examples should not be limited by the particular disclosed embodiments described above.

What is claimed is:

1. A method comprising:
measuring, by at least one gas sensor, gas concentrations at a plurality of locations;
transmitting, by the gas sensor, a plurality of concentration signals corresponding to the measured gas concentrations to a processor;
evaluating, by a feature identification module of the processor, each individual concentration signal of the concentration signals with a plurality of criteria, wherein the step for evaluating includes identifying if the individual concentration signal is a peak signal based on whether a plurality of values extracted from the individual concentration signal according to the plurality of criteria are within thresholds and tagging the individual concentration signal as a feature if the individual concentration signal is the peak signal; and
calculating, by an ambient background level calculator module of the processor, an ambient background gas level by removing the measured gas concentration corresponding to the concentration signal set as a feature from an original background level.

2. The method of claim 1, wherein the step for evaluating includes:
extracting, by a criteria value extraction module of the processor, a plurality of values from the individual concentration signal according to the plurality of criteria.

3. The method of claim 2, wherein the step for evaluating each individual concentration signal includes:
determining, by a value on and off setting module of the processor, if the plurality of values are within the thresholds for the plurality of criteria, respectively, and setting the values as on if the value is within a threshold;
generating, by an integer value generation module of the processor, an integer value of the individual concentration signal by adding up value contribution of all the values that are set as on; and
tagging, by a feature tagging module, the concentration signals as a feature or a no-feature, wherein a concentration signal is tagged as a feature if the integer value of the concentration signal is above a threshold.

4. The method of claim 3, wherein the step for evaluating each individual concentration signal is performed based on a feature bitmask including a plurality of bits by a feature mask processing module, and
wherein the values extracted from the individual concentration signal respectively corresponds to the plurality of bits in the feature bitmask.

5. The method of claim 4, wherein the value contribution by each bit in the feature bitmask is calculated in a binary number, which is represented as a power of 2.

6. The method of claim 4, wherein the values from the extracted from the individual concentration signal includes at least two of:
a concentration value;
a normalized power;
a normalized wavelet probability density function (PDF);
a centroid of a normalized wavelet cumulative distribution function (CDF);
a concentration rate-of-change;
a vertical velocity; and
a persistence.

7. The method of claim 6, wherein in the step for setting the values as on, the concentration value is set as on if the concentration value is above a predetermined threshold, the normalized power is set as on if the normalized power is above a predetermined threshold, the normalized wavelet PDF is set as on if the normalized wavelet PDF is above a predetermined threshold, the centroid of a normalized wavelet CDF is set as on if the centroid of a normalized wavelet CDF is above a predetermined threshold, the concentration rate-of-change is set as on if the concentration rate-of-change is above a predetermined threshold, the vertical velocity is set as on if the vertical velocity is below a predetermined threshold; and the persistence is set as on if an individual concentration signal directly before a current individual concentration signal is tagged as a feature.

8. The method of claim 6, wherein the feature bitmask is based on out of 7 bits, and wherein the seventh bit position is corresponding to a concentration value, the sixth bit position is corresponding to a normalized power; the fifth bit position is corresponding to a normalized wavelet probability density function (PDF); the fourth bit position is corresponding to a centroid of a normalized wavelet cumulative distribution function (CDF); the third bit position is corresponding to a concentration rate-of-change; the second bit position is corresponding to a vertical velocity; and the first bit position is corresponding to a persistence.

9. The method of claim 1, wherein the at least one gas sensor is mounted on an unmanned aerial system (UAS), and the step of measuring the gas concentrations at the plurality of positions is performed while the UAS is during the flight.

10. The method of claim 9, wherein the step for measuring the gas concentrations is performed at the plurality of locations over a cross section of a gas plume within a flux plane geometry.

11. The method of claim 1, wherein the step for measuring gas concentrations is performed repeatedly at the same locations in a regular basis.

12. The method of claim 1, further comprising: displaying, by a display, a normally-distributed surface of the measured gas concentrations.

13. A method comprising:
measuring, by at least one gas sensor, a gas concentration;
determining, by a feature mask processing module of a processor, if a concentration value from a concentration signal corresponding to the gas concentration is a feature;
determining, by the feature mask processing module, if a normalized power from the concentration signal is a feature;
determining, by the feature mask processing module, if a normalized wavelet probability density function (PDF) from the concentration signal is a feature;
determining, by the feature mask processing module, if a centroid of a normalized wavelet cumulative distribution function (CDF) from the concentration signal is a feature;
determining, by the feature mask processing module, if a concentration rate-of-change from the concentration signal is a feature;
determining, by the feature mask processing module, if a vertical velocity from the concentration signal is a feature; and
determining, by the feature mask processing module, if a persistence from the concentration signal is a feature.

14. The method of claim 13, further comprising:
generating a bitmask based on each determined feature, wherein a 1 indicates a feature and a 0 indicates no feature.

15. The method of claim 14, further comprising:
decoding a whole number into an on or off state of thresholds comprising the bitmask.

16. A system comprising:
at least one gas sensor configured to measure gas concentrations at a plurality of locations and transmit a plurality of concentration signals corresponding to the measured gas concentrations to a processor; and
the processor configured to communicate with the at least one gas sensor and including:
a feature identification module configured to evaluate each individual concentration signal of the concentration signals with a plurality of criteria by identifying if the individual concentration signal is a peak signal based on whether a plurality of values extracted from the individual concentration signal according to the plurality of criteria are within thresholds and tagging the individual concentration signal as a feature if the individual concentration signal is the peak signal; and
an ambient background level calculator module configured to calculate an ambient background gas level by removing the measured gas concentration corresponding to the concentration signal set as a feature from an original background level.

17. The system of claim 16, wherein the feature identification module includes:
a criteria value extraction module configured to extract a plurality of values from the individual concentration signal according to the plurality of criteria;
a value on and off setting module configured to determine if the plurality of values are within the thresholds for the plurality of criteria, respectively, and set the values as on if the value is within a threshold;
an integer value generation module configured to generate an integer value of the individual concentration signal by adding up value contribution of all the values that are set as on; and
a feature tagging module configured to tag the concentration signals as a feature or a no-feature, wherein a concentration signal is tagged as a feature if the integer value of the concentration signal is above a threshold.

18. The system of claim 17, wherein the feature identification module includes a feature mask processing module configured to generate a feature bitmask including a plurality of bit, and wherein the values extracted from the individual concentration signal respectively corresponds to the plurality of bits in the feature bitmask.

19. The system of claim 17, wherein the values from the extracted from the individual concentration signal includes: a concentration value, a normalized power, a normalized wavelet probability density function (PDF), a centroid of a normalized wavelet, cumulative distribution function (CDF), a concentration rate-of-change, a vertical velocity, and a persistence; and
wherein the concentration value is set as on if the concentration value is above a predetermined threshold, the normalized power is set as on if the normalized power is above a predetermined threshold, the normalized wavelet PDF is set as on if the normalized wavelet PDF is above a predetermined threshold, the centroid of a normalized wavelet CDF is set as on if the centroid of a normalized wavelet CDF is above a predetermined threshold, the concentration rate-of-change is set as on if the concentration rate-of-change is above a predetermined threshold, the vertical velocity is set as on if the vertical velocity is below a predetermined threshold; and the persistence is set as on if an individual concentration signal directly before a current individual concentration signal is tagged as a feature.

20. The system of claim 16, wherein the at least one gas sensor is mounted on an unmanned aerial system (UAS), and the gas concentrations at the plurality of positions is measured while the UAS is during the flight.
